SEQUENCE LISTING (2 sequences)

SEQ ID NO: 1 - protein, 7 aa, Artificial Sequence
  A conserved region in E protein domain II
RCPTTGE

SEQ ID NO: 2 - protein, 35 aa, Artificial Sequence
  multiple copies of a conserved region in E protein domain II
RCPTTGERCPTTGERCPTTGERCPTTGERCPTTGE

(19) United States
(12) Patent Application Publication
(10) Pub. No.: US 2021/0060152 A1
(43) Pub. Date: Mar. 4, 2021

(54) MULTIPLE MOSQUITO-BORNE FLAVIVIRUS VACCINE AND USE THEREOF IN INDUCING NEUTRALIZING ANTIBODIES

(71) Applicant: NATIONAL HEALTH RESEARCH INSTITUTES, Zhunan Township (TW)

(72) Inventors: CHING-LEN LIAO, Zhunan Township (TW); MING-SHU HSIEH, Zhunan Township (TW); SHU-PEI LIEN, Zhunan Township (TW)

(21) Appl. No.: 17/011,140

(22) Filed: Sep. 3, 2020

Related U.S. Application Data
(60) Provisional application No. 62/895,307, filed on Sep. 3, 2019.

(51) Int. Cl. A61K 39/12; C07K 14/005; A61P 31/14

(52) U.S. Cl. CPC A61K 39/12; C07K 14/005; A61K 2039/575; C12N 2770/24022; C12N 2770/24034; A61P 31/14

(57) ABSTRACT

The present invention relates to a peptide immunogen, comprising at least one copy of amino acid sequence RCPTTGE (called JBP). The peptide immunogen of present invention can induce an effective immune response to two or more mosquito-borne flavivirus. The present invention also provides mono/multivalent virus vaccines to protect animal against multiple mosquito-borne flavivirus infection, including diseases caused by Japanese encephalitis virus (JEV), West Nile virus (WNV), Dengue Virus (DENV), and Zika virus (ZIKV) infections.

### BACKGROUND OF THE INVENTION

### Technical Field of the Invention

The present invention relates to a broad-spectrum neutralizing vaccine against mosquito-borne flavivirus and a using method thereof in protecting animal against virus infection. More particularly, it relates to a use of one copy or multiple copies of a high conserved peptide sequence in the E protein of mosquito-borne flavivirus for manufacturing mono/multivalent or passive virus vaccine.

### Background

Mosquito-borne flaviviruses (genus Flavivirus, family Flaviviridae), such as Japanese encephalitis virus (JEV), West Nile virus (WNV), Dengue Virus (DENV) and Zika virus (ZIKV), are major infectious disease that expand public health problem in tropical and subtropical areas around the world. Except for JEV, there is still no vaccine available against WNV, DENVs or ZIKV. One current challenge for DENVs and ZIKV vaccine development is the presence of dominated cross-reactive antibodies that bind to but weakly neutralize the different serotypes of DENV or ZIKV. These cross-reactive antibodies not only are unbeneficial to induce cross-protection but may deteriorate disease by the phenomenon of antibody-dependent enhancement (ADE). The mechanism of ADE is well-documented and caused by the non-neutralizing or sub-neutralizing antibodies that bind with virus particles to form virus-antibody complexes and attach to cell surface by binding to Fcγ receptor (FcγR) of the cell surface through Fcγ chain of antibody, and then the virus-antibody complexes are internalized into cells that will lead to more serious infection. Therefore, ADE phenomenon is the major reason of the appearance of secondary infection, such as dengue fever (DF) or dengue hemorrhagic fever (DHF). However, the antibodies produced during the primary infection can recognize but not neutralize the other serotypes of secondary infecting virus. In the light of this, the new report from World Health Organization admonished that the currently licensed dengue vaccine from Sanofi company may have high safety concerns. And the higher risk of severe dengue infection occurred in the subset of trial participants who were inferred to be seronegative. Hence, above problem underlines the urgent need to develop a novel vaccine against multiple mosquito-borne flavivirus.

The genome of mosquito-borne flaviviruses is a single-stranded, positive-sense RNA which encodes the structural proteins capsid (C), membrane precursor (prM), envelope and the nonstructural proteins NS1, NS2, NS3, NS4, and NS5. The E glycoprotein is essential for cellular receptors binding and virus entry through cellular membrane fusion. In addition, the E glycoprotein is the major antigen that induces protective immunity, such as production of neutralizing antibodies. Based on the crystal structures, the E glycoprotein are divided into three functional domains: domains I, II and III. The domain III is the most variable amino acid sequence among flavivirus; hence, the antibodies targeting this domain are highly virus specific. Moreover, this domain contains the primary receptor-binding motifs, and there are many evidences proved by epitope mapping indicating that the domain III can induce stronger neutralizing antibodies. In contrast, domain II is conserved among flavivirus, so it is possible to induce cross-reactive antibodies. However, the neutralizing capacity of most antibodies induced by domain II tend to be weakly. For example, most of the antibodies targeting the conserved fusion loop on domain II of E glycoprotein are with low-potency neutralization activity and can induce significant antibody-dependent enhancing in vitro and in vivo. Therefore, other conserved regions, such as be loop, are proposed and suggested to have the potential for inducing stronger humoral immune response. Previous study finds a clone antibody, 1C19, which recognizes the bc loop of domain II and exhibits the ultrahigh neutralizing capacity for all four serotypes DENV. The critical residues on the DENV E protein for 1C19 binding are R73, G78 and E79, and these residues are found to be conserved in JEV, DENVs, and ZIKV as well.

Therefore, the present invention discloses that the region between amino acid residues 73 and 79 in E protein domain II (EDII₇₃₋₇₉) is highly conserved in each virus, and the EDII₇₃₋₇₉ region has the potential to be used as a potent epitope to induce the antibody that against JEV, DENVs, and ZIKV to be neutralized.

More specifically, the present invention attempts to use a designed amino acid sequence RCPTTGE (JEV bc loop peptide, JBP, SEQ ID No.1) in inducing high effective antibody response to multiple mosquito-borne flavivirus, such as for Japanese encephalitis virus (JEV), West Nile virus (WNV), Dengue Virus (DENV), and Zika virus (ZIKV).

### SUMMARY OF INVENTION

Based on the above objects, the present invention demonstrates that JBP is a immunogen with great ability to induce stronger humoral immune response and induce effective neutralizing antibodies against multiple mosquito-borne flavivirus.

Accordingly, one aspect of the invention relates to a peptide comprising at least one copy of amino acid sequence RCPTTGE (called JBP, SEQ ID No.1), which induce antibody response to two or more mosquito-borne flavivirus.

In some embodiments of the present invention, the peptide induces neutralizing antibodies against multiple mosquito-borne flavivirus in immunized animal.

In other embodiments of the present invention, the mosquito-borne flavivirus includes JEV, DENVs and ZIKV.

In another aspect of the present invention, it is related to a nucleotide sequence coding for a peptide comprising one or more copies of RCPTTGE.

Another aspect of the present invention relates to a method for protecting animal against mosquito-borne flavivirus infection, the method comprises immunizing the animal with a peptide immunogen comprising at least one copy of JBP.

In certain embodiments of the present invention, the peptide immunogen comprises an amino acid sequence having five copies of JBP (RCPTTGERCPTTGERCPTTGERCPTTGERCPTTGE, SEQ ID No.2).

A further aspect of the present invention relates to a method for controlling mosquito-borne flavivirus infection, the method comprises administration of antibodies induced by a peptide immunogen comprising at least one copy of JBP.

A further aspect of the present invention relates to a multiple mosquito-borne flavivirus vaccine, comprising a peptide having at least one copy of amino acid sequence RCPTTGE.

In some embodiments of the present invention, the multiple mosquito-borne flavivirus vaccine induces of an effective neutralizing antibody response to JEV, DENVs and ZIKV.

### BREIF DESCRIPTION OF THE DRAWINGS

FIG. 1 demonstrates the conserved region between residues 73 to 79 in E protein domain II.

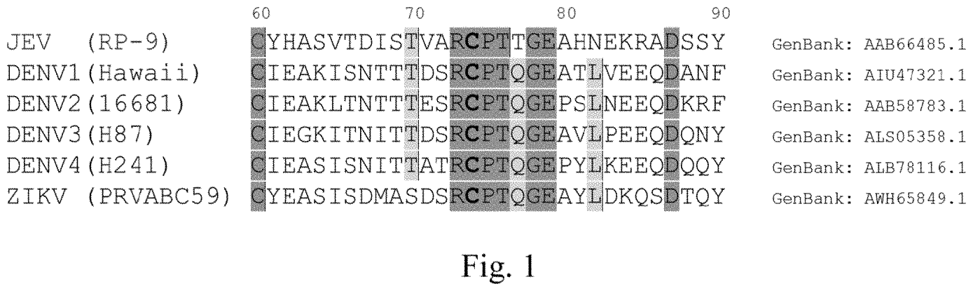

FIG. 2. shows the effects of the JBP in neutralizing antibody response against JEV, DENVs and ZIKV.

FIG. 3. shows the protective effect of JBP induced antibodies against JEV challenge.

FIG. 4. shows the protective effect of JBP induced antibodies against DENV-1 and DENV-2 challenge.

FIG. 5. illustrates the inhibition of viremia after challenge with DENV-4 and ZIKV.

### DETAILED DESCRIPTION OF THE INVENTION

Other features and advantages of the present invention will be further exemplified and described in the following examples, which are intended to be illustrative only and not to limit the scope of the invention.

As used herein, the term “animal” has its ordinary meaning and includes, but not limited to, a bird, a fish and a mammal. In some embodiments, the mammal comprises, but not limited to, dogs, cats. horses, sheep, rodents and primates, including human.

### EXAMPLE 1

### Production of Neutralizing Antibodies Against Multiple Tick-Borne Flaviviruses After JBP Vaccination

The neutralizing capacity of the antibodies induced by JBP vaccination is evaluated. BALB/c mice are divided into three groups (6 mice per group) and immunized with 30 μg 1× JBP, 5× JBP or PBS by the subcutaneous injection respectively, and then all the mice are boosted two times with a two-week interval during immunization. Sera from individual BALB/c mice were collected at the sixth week after first vaccination. Sera were pooled and serially diluted from 1:8 to 1:512 with PBS. The neutralizing titer of the immunized mice sera against JEV, four serotypes of DENVs, and ZIKV is assessed by focus reduction neutralization tests (FRNT₅₀).

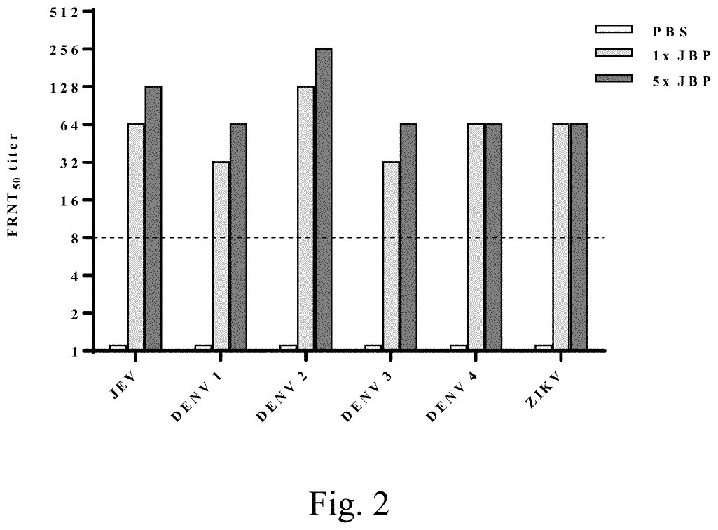

The results indicate that 5× JBP-immunized sera exhibits higher FRNT₅₀ titers ranging from 64 to 256 and 1× JBP-immunized sera shows moderate FRNT₅₀ titers ranging from 32 to 128, and both are significantly higher than those of the control PBS-immunized sera (FIG. 2), which indicates that JBP can induce the neutralizing antibodies against JEV, DENVs and ZIKV.

### EXAMPLE 2

### Induction of Prospective Antibodies Against Japanese Encephalitis Virus and Dengue Virus in Animal Model

The protective effect of 1× and 5× JBP-immunized sera against JEV is examined. The IgG purified from pre-immunized sera, 1× or 5× JBP-immunized sera is respectively adoptive transferred to ICR mice and the mice are challenged with 1×10⁵ PFU of JEV by the intraperitoneal plus intracardiac route on day 1 post transfer.

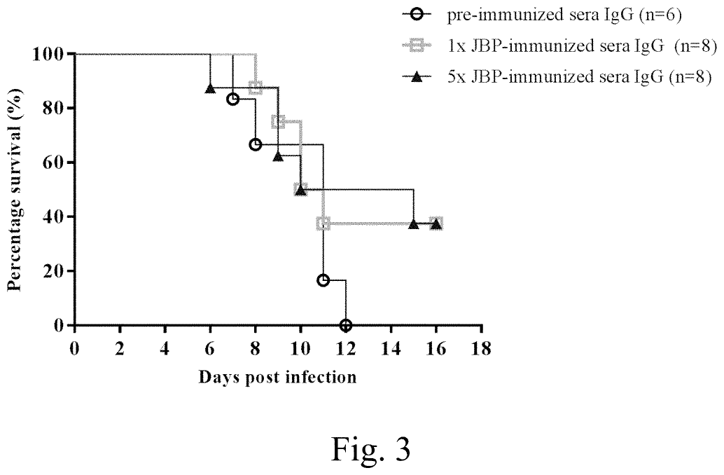

As showed in FIG. 3, the 1× and 5× JBP-immunized sera IgG-transferred mice exhibit the higher survival rates (37.5%) after 30 days post infection; however, all the pre-immune sera IgG-transferred mice are dead within 12 days post infection. The results suggest that JBP vaccination can induce the production of the IgG with the protective effect against JEV challenge.

Furthermore, the protective effect of JBP-immunized sera against JEV is examined as well. Six-week-old AG129 mice are intraperitoneally (intraperitoneal injection) transferred with 50 μg ×, 5× JBP-immunized sera IgG or 50 μg pre-immune sera IgG as a control. The IgG transferred mice are challenged with 2.65×10⁸ FFU of DENV-2 or 1×10⁷ FFU of DENV-1 by intraperitoneal injection on day 1 post transfer. The mice are monitored for mortality for 60 days.

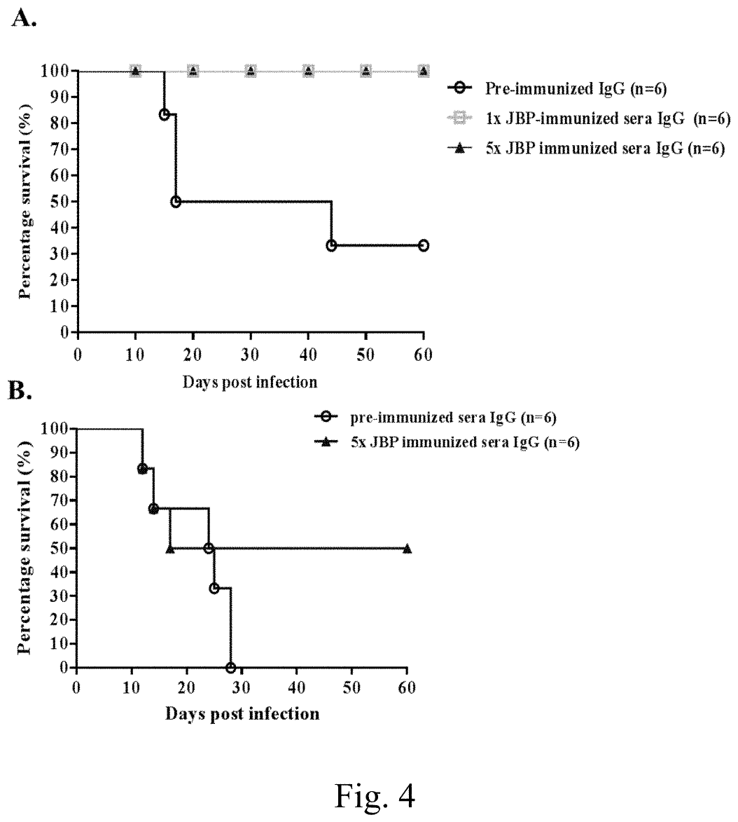

Likewise, mice transferred with 1× or 5× JBP-immunized sera IgG possess complete protective effect against DENV-2 challenge (100% survival rate). In contrast, mice transferred with pre-immunized sera IgG have lower survival rate (FIG. 4A). On the other hand, the mice transferred with 5× JBP-immunized sera IgG also can defense DENV-1 infection with 50% survival rate when the mice of control group are all dead (FIG. 4B). Overall, the results indicate that 5× JBP-immunized sera IgG provide the protective effect against DENV-1 and DENV-2 challenge.

### EXAMPLE 3

### The inhibition of Type IV Dengue Virus and Zika Virus Viremia by JBP-Induced Antibodies

The protective effect against DENV-4 and ZIKV of the sera IgG induced by 5× JBP immunization is examined. Six-week-old AG129 mice are transferred with 1 μg, 10 μg, or 50 μg 5× JBP-immunized sera IgG or 50 μg pre-immunized sera IgG by intraperitoneal injection. The IgG transferred mice are challenged with 2×10⁷FFU of DENV-4 or 10 FFU ZIKV by the intraperitoneal route on day 1 post transfer. The survival rates of these mice were monitored daily. Viral load in the sera after DENV-4 or ZIKV challenge is determined by focus forming assay on day 3 post challenge.

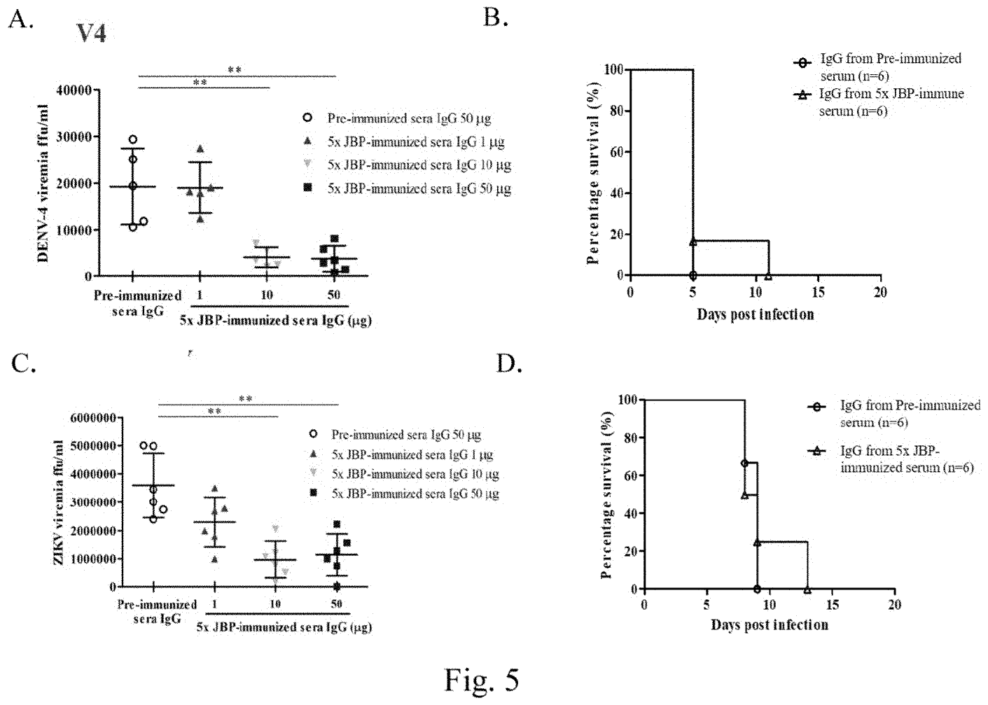

As shown in FIG. 5, comparing with the mice treated with 50 μg pre-immunized sera IgG, the viremia levels of the mice treated with 5× JBP-immunized sera IgG are significantly decreased after DENV-4 or ZIKV challenge (FIGS. 5A and 5C). The inhibition effect of 5× JBP-immunized sera IgG is dose-dependent; however, the dose of 10 μg and 50 μg 5× JBP-immunized sera IgG shows the similar inhibition effect. Therefore, the dose of 5× JBP-immunized sera IgG in further survival test is chosen as 10 μg 5× JBP-immunized sera IgG.

In the further survival test, no mice survive in any group after DENV-4 or ZIKV challenge, but the mice treated with 10 μg 5× JBP-immunized sera IgG live longer than the mice treated with 10 μg pre-immunized sera IgG (FIGS. 5B and 5D). The results indicate that 5× JBP immunization induce the production of protective antibodies against DENV-4 and ZIKV challenge to reduce viremia levels.

In summary, the present invention first discloses an amino acid sequence conserved in the E protein domain II of mosquito-borne flavivirus, and designs a neutralizing epitope RCPTTGE (named JBP) to be developed as the efficacious multivalent mosquito-borne flaviviruses vaccine and as the efficacious antigen to product anti-virus antibody.

1. A peptide immunogen, comprising at least one copy of amino acid sequence RCPTTGE (SEQ ID No.1), wherein the peptide induces an antibody in response to two or more mosquito-borne flavivirus.

2. The peptide immunogen of claim 1, wherein the peptide induces neutralizing antibodies against multiple mosquito-borne flavivirus in immunized animal.

3. The peptide immunogen of claim 2, wherein the mosquito-borne flavivirus comprises at least one flavivirus selected from Japanese encephalitis virus (JEV), West Nile virus (WNV), Dengue Virus (DENV), and Zika virus (ZIKV).

4. An isolated nucleotide sequence, coding for the peptide immunogen of claim 1.

5. A multiple mosquito-borne flavivirus vaccine, comprising the peptide immunogen of claim 1.

6. The multiple mosquito-borne flavivirus vaccine of claim 5, which induces an effective neutralizing antibody response to at least one flavivirus selected from Japanese encephalitis virus (JEV), West Nile virus (WNV), Dengue Virus (DENV), and Zika virus (ZIKV).

7. The multiple mosquito-borne flavivirus vaccine of claim 5, which induces protecting antibodies against JEV, DENVs and ZIKV.

8. A method for protecting animal against mosquito-borne flavivirus infection, which comprises immunizing the host in need with the peptide immunogen of claim 1.

9. The method of claim 8, the peptide immunogen comprises an amino acid sequence of RCPTTGERCPTTGERCPTTGERCPTTGERCPTTGE (SEQ ID No.2).

10. A method for controlling mosquito-borne flavivirus infection, which comprises administration of antibodies induced by the peptide immunogen of claim 1.
